(19) United States
(12) Patent Application Publication
(10) Pub. No.: US 2006/0249145 A1
(43) Pub. Date: Nov. 9, 2006

(54) Storage, homogenisation and dosage system

(75) Inventors: Gianpaolo Fasoli, Milan (IT); Mauro Carraro, Varese (IT); Mauro Fassi, Milan (IT); Flavio Crestetto, Milan (IT)

Correspondence Address: HEDMAN & COSTIGAN P.C., 1185 AVENUE OF THE AMERICAS, NEW YORK, NY, 10036, US

(73) Assignee: CS AUTOMAZIONE S.r.l.

(21) Appl. No.: 11/403,793

(22) Filed: Apr. 13, 2006

(30) Foreign Application Priority Data
May 9, 2005 (IT) MI2005A 000829

(51) Int. Cl. A61M 11/00

(52) U.S. Cl. 128/200.23

(57) ABSTRACT

Storage, homogenisation and dosage system (10) for substances susceptible to precipitation or aggregation, including at least one tank (20) for storage and at least one valve (40) for the dosage of said substances, the at least one tank (20) comprises a series of portions (54, 64) protruding inside the at least one tank (20), which are made integral with the same by means of fixing means, the system (10) also comprises supporting and rotation means (30) for allowing a good homogenisation of the substances contained in the at least one tank (20).

The present invention relates to a storage, homogenisation and dosage system, which can be used for substances susceptible to precipitation or aggregation, such as, in particular, dyeing substances in a liquid or solid dispersion.

Dyeing substances, individually dosed in suitable proportions, contribute to the preparation of final colours, which are then used in many applicative fields.

Dyeing substances comprise organic or inorganic pigments, which can be used directly in solid form or can be dispersed, in water or other solvents, before use.

In the case of dyeing substances used in liquid form, these are previously stocked into tanks which feed, by means of a hydraulic circuit, a system for the dosage of the same.

In the automatic dosage of dyeing substances, in any applicative field, the final colour result is heavily influenced by the concentration of the raw materials used.

The precision of effecting the measures by weight or volume, for a correct dosage of the dyeing substances, is therefore of great importance as is also maintaining, as much as possible, a perfect homogenisation of the dyeing substances.

In the case of dyeing substances mixed with one or more solvents or carriers, the solid particles, due to stratification and sedimentation phenomena, as they are heavier than the carrier in which they are suspended, are compelled, by the force of gravity, to deposit towards the lower point of the plant, i.e. towards the bottom of the tank and along the vertical tracts of the adduction tubes of the product to the dosage system, up to the inside of the dosage valves.

Consequently, in the absence of suitable expedients, the lowest parts of the plant tend to accumulate raw material which is more concentrated than the upper zones.

This generates, at the best, the handling, possibly extremely precise, of portions of dyeing substances having a concentration which varies with time.

At the beginning of the process, a sufficiently homogeneous dyeing substance is collected, whereas, with the passing of time and with the appearance of sedimentation phenomena, a dyeing substance richer in pigment is first collected (accumulated in the lower areas), and subsequently a dyeing substance impoverished by the same phenomenon.

As a consequence, with the same the amount of dyeing compound dosed, the dyeing capacity and therefore the possible shades resulting from their blends, are subject to variations, beyond control.

At worst, the extent of the phenomenon and the nature of the dyeing substance deposited, can cause the blockage of the system, in addition to serious and irreversible damage to the plant.

Taking this into account, suitable surfactant chemical products, called dispersers or suspension-aids, having a more or less distinct stabilizing effect on the suspensions are added to solutions containing dyeing substances with solvents.

In order to further improve the present situation, there is a tendency to increase the viscosity of the dyeing materials, up to the limits allowed by the subsequent utilization phases.

In both cases, the problem is neither radically nor definitely solved and consequently plant manufacturers must face and solve an uncontrolled and non-standardized situation.

In order to do this, they normally use homogenising systems based on the use of stirrers (to prevent sedimentation inside storage tanks) and pumps and recycling circuits which are as extensive as possible (to avoid the phenomenon which also takes place inside the tubes and valves) and also powerful (to avoid pressure drops due to the increase in viscosity of the raw materials and to the extension of the hydraulic supply circuits for delivery to dosage and return to the storage tank).

Some systems are equipped with a low-speed anchor stirrer, operated by means of a motoreducer.

These systems allow the suspension inside the storage tank of the raw material to be moved.

One of the disadvantages is that their use is possible only in the case of almost continuous use and therefore requires considerable energy costs.

Furthermore, these systems cannot be used in the case of very extensive feeding circuits.

Other systems are equipped with turbo-emulsifiers for high viscosity products, which involve a considerable high viscosity mass.

One of the disadvantages of these systems is that some particularly delicate dispersions can be irreversibly damaged by an excessive mechanical action.

Furthermore, not even these systems can be used in the case of very large feeding circuits and in the case of non-continuative use. Other systems are equipped with complex hydraulic systems, with delivery-and-return lines and temporised recycling pumps.

These systems allow an optimum homogenisation inside the tubes and, by means of a series of expedients, also inside the storage tanks and dosage valves.

A disadvantage of these systems is that they require higher costs, both in terms of energy requirement and in terms of time for the maintenance of the components of the hydraulic circuit.

A disadvantage of the systems described is that they involve a considerable cost increase, represented, for example, by a higher investment cost of the plant for double hydraulic circuits and/or for the assembly of the stirrers.

Another disadvantage of these systems is that they are complicated, due to the increase in the number of dynamic components, such as pumps, stirrers, integral recycling pumps.

Another disadvantage is that as the dynamic components are subject to wear, they require periodical maintenance, which is onerous due to time consumption and costs.

A further disadvantage of these systems is that they require a considerable increase in energy consumption for running the plant.

There can also be problems in the case of dyeing substances, or, in any case, reagents used directly in solid form, relating to blockage of the system and damage to the plant caused by aggregation phenomena of the solid particles into agglomerates, both inside the tanks and in other parts of the plant.

For this reason, the tanks envisage kinematisms in their interior, capable of breaking the aggregates to maintain the material finely dispersed.

An objective of the present invention is to provide a storage, homogenisation and dosage system which solves the drawbacks of the known systems.

Another objective is to provide a storage, homogenisation and dosage system for substances susceptible to precipitation and/or aggregation, which is simple and economical.

Yet another objective is to avail of a storage, homogenisation and dosage system, which can be used for dyeing substances dispersed in a solvent and for solid dyeing substances.

These objectives according to the present invention are achieved by providing a storage, homogenisation and dosage system as disclosed in claim 1.

Further characteristics of the invention are indicated in the subsequent claims.

The characteristics and advantages of a storage, homogenisation and dosage system according to the present invention will appear more evident from the following illustrative and non-limiting description, referring to the enclosed schematic drawings in which:

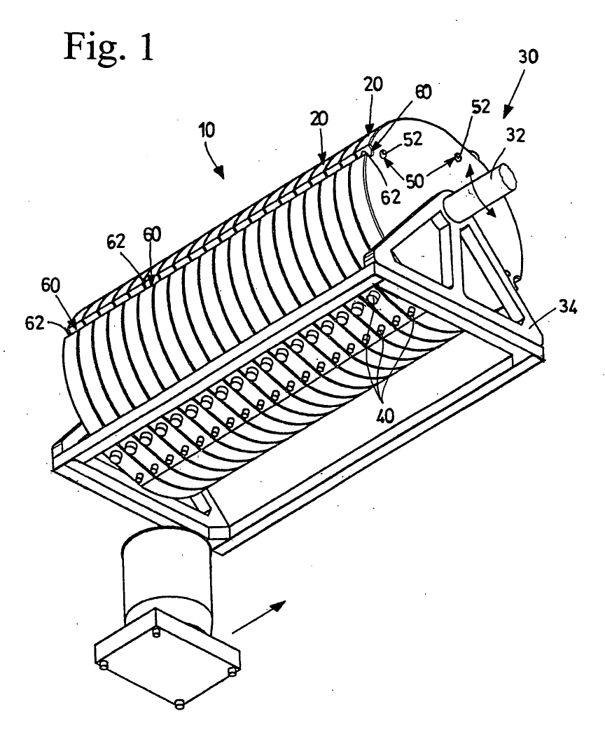

FIG. 1 is a raised, perspective, right-side view from below, which shows a preferred embodiment of a storage, homogenisation and dosage system according to the present invention;

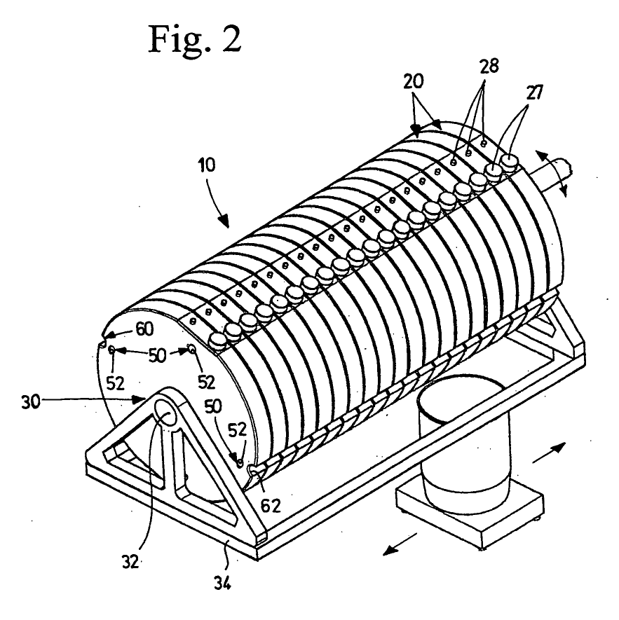

FIG. 2 is a raised, perspective, left-side view from above of the storage system of FIG. 1;

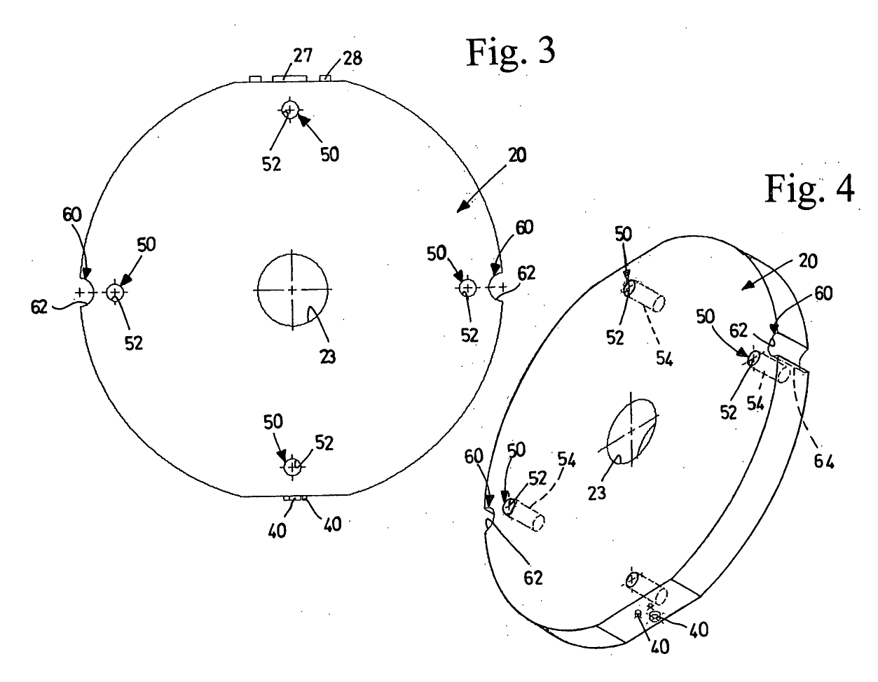

FIG. 3 is a plan view of a preferred embodiment of a tank in a storage, homogenisation and dosage system according to the present invention;

FIG. 4 is a perspective, raised side-view from above the tank of FIG. 3.

With reference to the figures, these illustrate a storage, homogenisation and dosage system, indicated as a whole with 10, of substances or dispersions susceptible to precipitation and/or aggregation and/or sedimentation, such as, in particular, organic and/or inorganic dyeing substances.

Said system 10 comprises at least one storage tank 20, at least one dosage valve 40 of said substances and supporting and rotation means 30.

The at least one tank 20 comprises a series of portions (54,64) protruding inside said at least one tank 20, which are made integral with the same by fixing means to allow a good homogenisation of said substances contained in said at least one tank 20.

Each of the protruding portions (54,64) is a portion of a protruding element (50,60) made integral with or in a single piece with said at least one tank 20, which is capable of generating a mixing of the dyeing substances or, in any case, susceptible to aggregation and sedimentation when a preferably rotatory movement is conferred to said at least one tank 20, by the supporting and rotation means 30.

Each of the protruding elements (50,60), which protrudes internally in said at least one tank 20, is preferably a peg and/or a protuberance and/or a tubular element and/or a tubular portion or similar element.

Said at least one tank 20 preferably comprises a series of protruding elements (50,60) each of which having an external portion (54,64) protruding internally in said at least one tank 20 and an internal portion (52,62) which is external to said at least one tank 20 or on the external surface of said at least one tank 20.

In other words, each protruding element (50,60) preferably has an external portion (54,64) which is internal in said at least one tank 20 for stirring said substances and also an inner portion (52,62) having a further function.

Each protruding element (50,60) is equipped with an internally protruding portion (54,64) and integral with said at least one tank 20.

In particular, according to a preferred embodiment of the present invention, said at least one tank 20 comprises a series of tubular elements 50, each of which has a portion or external surface 54 which is internal to said at least one tank 20 and preferably having the function of causing a mixing of said dyeing substances when said at least one tank 20 is put under rotation by the action of supporting and rotation means 30.

In other words, said at least one tank (20) comprises a series of tubular elements (50) each of which comprising a portion internally protruding (54) inside said at least one tank (20).

Furthermore, each tubular element 50 preferably has an internal portion 52 (innermost with respect to the external portion 54) which is external to said at least one tank 20, or, in any case, is on the external surface of said at least one tank 20, and preferably acting as a housing for a tie rod or a pole, not shown, so that at least two tanks 20 can be packed or fixed to each other.

Each tubular element 50 preferably also has a reinforcing function for said at least one tank 20.

According to a further preferred embodiment of the present invention, said at least one tank 20 comprises a series of reinforcing elements 60 preferably situated near the end portions of the same so as to stiffen its structure making it more solid.

Each reinforcing element 60 has a portion or external surface 54 which is internal to said at least one tank 20 and preferably having the function of causing a mixing of said dyeing substances when said at least one tank 20 is rotated by driving the supporting and rotation means 30.

In other words, said at least one tank (20) comprises a series of reinforcing elements (60) each of which comprising an internally protruding portion (64) in said at least one tank (20).

Furthermore, each reinforcing element 60 preferably has an internal portion 62 which is outside said at least one tank 20 and has the function of stiffening its structure and also acting as a housing for a pole, not shown, so at to contemporaneously confer the same rotating movement to at least two tanks 20 fixed to each other.

The at least one tank 20 preferably also comprises a stopper 27 for introducing substances to be stored into said at least one tank 20, and a valve 28 for the entry or discharge of air to allow a better dosage of the dyeing substances.

Said at least one dosage valve 40, moreover, is preferably housed near a peripheral portion of said at least one tank 20.

The supporting and rotation means 30 preferably comprise a fixed structure or frame 34 to which said at least one tank 20 is preferably hinged.

Furthermore, the supporting and rotation means 30 preferably comprise a shaft 32 for supporting and rotating said at least one tank 20.

Said at least one tank 20 is preferably hinged to supporting and rotation means 30 by means of said shaft 32.

In particular, said at least one tank 20 comprises a seat 23 in which said shaft 32 is inserted.

Said seat 23 is preferably a pass-through hole arranged centrally with respect to said at least one tank 20.

This allows an easy and rapid rotation of said at least one tank 20.

By collaborating with said series of protruding portions (54, 64), said supporting and rotation means 30 cause a turbulent movement or stirring of said substances contained in said at least one tank 20, consequently avoiding aggregation and/or precipitation phenomena and/or variations in the concentration of said substances.

In other words, it is possible keep the concentration of said substances contained in said at least one tank 20, in suspension and/or homogenise them by means of a series of protruding portions (54, 64) and/or protuberances and/or similar devices arranged internally and integral with said at least one tank and by means of said supporting and rotation means 30 envisaged for rotating and/or oscillating and/or tilting said at least one tank 20.

Furthermore, the shaft 32 of the supporting and rotation means 30 is capable of rotating with respect to a longitudinal axis of the same to consequently allow said at least one tank 20 to rotate or oscillate, as it is hinged to the fixed structure 34, preferably by means of hinges.

In this way, it is possible to homogenise the substances contained inside the at least one tank 20, whether they be solid or liquid.

To enable this, the shaft 32 is connected and is activated by means of a motor driven by a processing unit, not shown in the figures.

Furthermore, said supporting shaft 32 has a shaped section which is preferably circular or square or polygonal.

The activation of the supporting and rotation means 30 can be effected either continuously or non-continuously, so as to allow continuous usage of the substances contained inside the at least one tank, whether they be liquid or solid, pure substances or mixtures, but in any case susceptible to precipitation or aggregation.

In this way, it is possible to integrate the storage and dosage functions in a single unit (the at least one tank 20).

It is possible, in fact, to rotate the at least one tank 20 by means of supporting and rotation means 30 so that said at least one valve 40 is facing downwards or, in other words, in a dosage position.

The substances contained inside said at least one tank 20 can be tapped by means of a suitable dosage device or system, by activating the dosage valve 40.

The dosage device is capable of opening the dosage valve 40 and is also capable of introducing air through the valve 28 so as to pour, in a controlled manner, a well-defined quantity by weight or by volume of the substances contained in the at least one tank 20 into a container positioned below the dosage valve 40.

The storage homogenisation and dosage system is particular suitable for being used for dyeing substances, either solid or in liquid form.

It is evident, however, that a dispersion or liquid solution or solid substance susceptible to causing problems of sedimentation and/or aggregation, can be stored inside the at least one tank 20.

A system according to the present invention is advantageously free of both advance and return adduction circuits thus avoiding the problems of the known art relating to precipitation inside the circuits themselves.

According to a further aspect of the present invention, a method is provided for the storage, homogenisation and dosage of substances, preferably dyes, susceptible to aggregation and/or precipitation, contained inside a tank 20 comprising a series of portions (54, 64) protruding inside said storage tank (20) internally integral therewith.

Said method envisages continuously or non-continuously rotating and/or undulating and/or oscillating said at least one tank 20 so as to mix and subsequently homogenise said dyeing substances by the combined action of said series of internally protruding portions (54, 64).

This is to avoid the precipitation and/or aggregation of said dyeing substances.

By preferably rotating said tank 20 turbulences are created due to the presence in its interior of a series of internally protruding portions (54, 64) which stir said dyeing substances without degrading their properties.

Said method is preferably applied to at least one tank 20 of the type described above.

It can thus be seen that a storage, homogenisation and dosage system according to the present invention achieves the objectives specified above.

The storage, homogenisation and dosage system of the present invention thus conceived can undergo numerous modifications and variations, all included in the same inventive concept.

Furthermore, in practice, the materials used, as also the dimensions and components, can vary according to technical demands.

1. A storage, homogenisation and dosage system (10) for substances susceptible to precipitation or aggregation, including at least one tank (20) for storage and at least one valve (40) for the dosage of said substances, characterized in that said at least one tank (20) comprises a series of protruding portions (54, 64) inside said at least one tank (20), which are made integral with the same by means of fixing means and in that it comprises supporting and rotation means (30) for allowing a good homogenisation of the substances contained in said at least one tank (20).

2. The system (10) according to claim 1, characterized in that each protruding portion (54, 64) is a portion of a protruding element (50, 60) integral with or in a single piece with said at least one tank (20) which is capable of generating a mixing of said substances when a movement is conferred by means of said supporting and rotation means (30).

3. The system (10) according to claim 1, characterized in that each protruding element (50, 60), which protrudes inside said at least one tank (20), is a peg and/or protuberance and/or tubular element and/or tubular portion or a similar element.

4. The system (10) according to claim 2, characterized in that each protruding element (50, 60), which protrudes inside said at least one tank (20), is a peg and/or protuberance and/or tubular element and/or tubular portion or a similar element.

5. The system (10) according to claim 1, characterized in that said at least one tank (20) comprises a series of protruding elements (50, 60) each of which having an external portion (54, 64) protruding inside said at least one tank 20 and an internal portion (52, 62) which is outside said at least one tank (20) or on the outer surface of said at least one tank (20).

6. The system (10) according to claim 1, characterized in that said at least one tank (20) comprises a series of tubular elements (50) each of which comprises a protruding portion (54) inside said at least one tank (20).

7. The system (10) according to claim 6, characterized in that each tubular element (50) comprises an internal portion (52) which is outside said at least one tank (20) and preferably acting as a housing for a tie-rod or pole.

8. The system (10) according to claim 1, characterized in that said at least one tank (20) comprises a series of reinforcing elements (60).

9. The system (10) according to claim 8, characterized in that said series of reinforcing elements (60) is arranged close to the end portions of said at least one tank (20).

10. The system (10) according to claim 8, characterized in that each reinforcing element (60) comprises a portion (64) protruding inside said at least one tank (20).

11. The system (10) according to claim 9, characterized in that each reinforcing element (60) comprises a portion (64) protruding inside said at least one tank (20).

12. The system (10) according to claim 8, characterized in that each reinforcing element (60) comprises an internal portion (62) which is outside said at least one tank (20) and having the function of stiffening its structure and also of acting as a housing for a pole.

13. The system (10) according to claim 1, characterized in that said at least one tank (20) comprises a stopper (27) for the introduction into the at least one tank (20) of substances to be stored and a valve (28) for the entry or discharge of air for a better dosage of said substances.

14. The system (10) according to claim 1, characterized in that said at least one dosage valve (40) is housed near a peripheral portion of said at least one tank (20).

15. The system (10) according to claim 1, characterized in that said supporting and rotation means (30) comprise a fixed structure or frame (34) to which said at least one tank (20) is preferably hinged.

16. The system (10) according to claim 1, characterized in that said supporting and rotation means (30) comprise a shaft (32) for supporting and rotating said at least one tank (20).

17. The system (10) according to claim 1, characterized in that said at least one tank (20) is hinged to said supporting and rotation means (30).

18. The system (10) according to claim 1, characterized in that said at least one tank (20) comprises a seat (23).

19. The system (10) according to claim 18, characterized in that said seat (23) is a pass-through hole arranged centrally with respect to said at least one tank (20) in which said shaft (32) is inserted.

20. The system (10) according to claim 16, characterized in that it comprises a motor device and a processing unit.

21. A method for the storage, homogenisation and dosage of substances susceptible to aggregation and/or precipitation contained inside a tank 20 comprising a series of portions (54, 64) protruding inside said storage tank (20) internally integral therewith, characterized in that said method envisages rotating and/or undulating and/or oscillating continuously and/or non-continuously said at least one tank 20 so as to mix and subsequently homogenise said substances by means of a combined action of said series of internally protruding portions (54, 64).

22. The method for the storage, homogenisation and dosage of substances susceptible to aggregation and/or precipitation contained inside a tank (20) according to claim 21, characterized in that said method envisages rotating said tank (20) causing turbulences due to the presence in its interior of said series of internally protruding portions (54, 64) which stir said substances without degrading their properties.
